(19) United States
(12) Patent Application Publication
(10) Pub. No.: US 2018/0142025 A1
(43) Pub. Date: May 24, 2018

(54) DIAGNOSTIC TEST INVOLVING ANTI-CD25-ADC

(71) Applicants: MedImmune Limited, CAMBRIDGE (GB); ADC THERAPEUTICS SA, EPALINGES (CH)

(72) Inventor: Patricius Henrikus Cornelis VAN BERKEL, EPALINGES (CH)

(21) Appl. No.: 15/571,969

(22) PCT Filed: Nov. 25, 2015
(86) PCT No.: PCT/EP2015/077683

§ 371 Date: Nov. 6, 2017

(30) Foreign Application Priority Data
May 7, 2015 (GB) 1507827.2

(51) Int. Cl. C07K 16/28; A61K 47/68; G01N 33/574; C07K 16/30; A61P 35/00

(52) U.S. Cl. CPC C07K 16/2866; A61K 47/6849; A61K 47/6803; G01N 33/57426; G01N 2800/7028; A61P 35/00; G01N 2333/70596; G01N 2800/50; G01N 2800/52; C07K 16/30

(57) ABSTRACT

The present disclosure relates to determining the eligibility of subjects for treatment and particularly, although not exclusively, to selecting subjects for treatment with an Antibody Drug Conjugate comprising an anti-CD25 antibody.

### FIELD

The present disclosure relates to determining the eligibility of subjects for treatment and particularly, although not exclusively, to selecting subjects for treatment with an Antibody Drug Conjugate comprising an anti-CD25 antibody.

### BACKGROUND

Antibody therapy has been established for the targeted treatment of subjects with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumor cells in the treatment of cancer, targets delivery of the drug moiety to tumors, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9):1087-1103; Payne, G. (2003) Cancer Cell 3:207-212; Trail et al (2003) Cancer Immunol. Immunother. 52:328-337; Syrigos and Epenetos (1999) Anticancer Research 19:605-614).

Maximal efficacy with minimal toxicity is sought thereby. Efforts to design and refine ADC have focused on the selectivity of monoclonal antibodies (mAbs) as well as drug mechanism of action, drug-linking, drug/antibody ratio (loading), and drug-releasing properties (Junutula, et al., 2008b Nature Biotech., 26(8):925-932; Dornan et al (2009) Blood 114(13):2721-2729; U.S. Pat. No. 7,521,541; U.S. Pat. No. 7,723,485; WO2009/052249; McDonagh (2006) Protein Eng. Design & Sel. 19(7): 299-307; Doronina et al (2006) Bioconj. Chem. 17:114-124; Erickson et al (2006) Cancer Res. 66(8):1-8; Sanderson et al (2005) Clin. Cancer Res. 11:843-852; Jeffrey et al (2005) J. Med. Chem. 48:1344-1358; Hamblett et al (2004) Clin. Cancer Res. 10:7063-7070). Drug moieties may impart their cytotoxic and cytostatic effects by mechanisms including tubulin binding, DNA binding, proteasome and/or topoisomerase inhibition. Some cytotoxic drugs tend to be inactive or less active when conjugated to large antibodies or protein receptor ligands.

The disclosure relates to the identification of subjects eligible for treatment with an antibody drug conjugate.

### SUMMARY

The methods described herein are useful for identifying a group of subjects who would benefit from treatment with an Antibody Drug Conjugate comprising an anti-CD25 antibody (an anti-CD25-ADC). In some aspects, the Antibody Drug Conjugate comprises an anti-CD25 antibody and a pyrrolobenzodiazepine dimer (a CD25-PBD-ADC). It has been previously shown that anti-CD25-ADCs are useful for treating CD25 expressing cancers (see, for example, WO2014/057119). Identification of a responder population (i.e. subjects with CD25 positive cells in the tumor microenvironment) will enhance the benefit-risk relationship of anti-CD25-ADCs, such as ADCT-301.

Provided herein are methods for determining whether or not a subject is suitable for treatment with anti-CD25-ADC. Also provided are methods for selecting a subject for treatment with an anti-CD25-ADC. In such methods, the expression of CD25 is detected in a sample obtained from the subject, and the subject is determined to be suitable for treatment with, or selected for treatment with, the anti-CD25-ADC.

In certain aspects, detecting expression of CD25 includes determining the level of CD25 expression in the sample. The level of CD25 may be determined quantitatively or semi-quantitatively. A subject may be determined to be suitable for treatment, or selected for treatment, if the level of CD25 is elevated or overexpressed in the sample. In some cases, the level of CD25 is determined relative to a control. The control may be a control sample of the same type of tissue as the sample, but from a subject who is known to be suitable for treatment with an anti-CD25-ADC, or from a subject who is known to not be suitable for treatment with an anti-CD25-ADC. The sample may be a sample of lymphoid tissue, such as a lymph node biopsy. The control sample may also be derived from cell lines known to express CD25 at a certain level. In certain aspects, a subject is determined to be suitable for treatment with an anti-CD25-ADC if 10% or more of the cells in the sample express CD25. In certain aspects, a subject is determined to be suitable for treatment with an anti-CD25-ADC if 10% or more of the tumor associated non-tumor cells in a sample express CD25. The tumor associated non-tumour cells may be lymphocytes.

The level of CD25 expression may be determined by immunohistochemistry or by another immunological technique. CD25 in the sample may be detected by binding to an antibody. The antibody is an anti-CD25 antibody. The antibody may be antibody 4C9. 4C9 antibodies are obtainable from Cell Marque antibodies by Ventana Medical Systems, Inc, Catalogue Number 760-4439. In other cases, the sample is a frozen sample. The sample may be prepared with, and stained with the antibody using a Ventana™ BenchMark ULTRA™ platform, Ventana™ Discovery™, Dako™ Omnis™, Dako™ AutostainerLink48™, Leica™ BOND RX™, Leica™ BOND-III™ or Leica™ BOND MAX™. In certain aspects, CD25 in the sample is indirectly detected, by binding an anti-CD25 antibody to the sample, and then binding a labelled antibody to the anti-CD25 antibody. Where the anti-CD25 antibody is a murine antibody, the labelled antibody may be an anti-mouse antibody. The labelled antibody may be labelled with horseradish peroxidase.

In certain aspects, detecting expression of CD25 includes grading the sample. The sample may be assigned a grade that is indicative of the level of CD25. The grade may be indicative of the severity of the disease. The grading may be made on the basis of all of the cells in the sample, or on the basis of a subset of such cells, for example, expression in the lymphocytes and/or Reed-Sternberg cells only. The grading of the sample may be dependent on the level of CD25 expression or the localization of the CD25 expression. The grading may additionally involve the analysis of other factors, such as the shape, size, type or appearance of cells in the sample, or the percentage of a particular type of cell in the sample. In some aspects, a subject is determined to be suitable for treatment, or is selected for treatment, if 10% or more of the cells in the sample is determined to exhibit grade 1 staining for CD25. In some cases, a subject is determined to be suitable for treatment, or is selected for treatment if 10% or more of the cells in the sample is determined to exhibit grade 2 staining for CD25. In some cases, a subject is determined to be suitable for treatment, or is selected for treatment if 1% or more of the cells in the sample is determined to exhibit grade 1 staining for CD25. In some cases, a subject is determined to be suitable for treatment, or is selected for treatment, based on the level of more than one tissue grade, such as the percentage of grade 1 staining and the percentage of grade 2 staining.

In some aspects, the dosage of anti-CD25-ADC is selected based on the level or pattern of CD25 expression observed. Thus, a higher expression of CD25, or the presence of a higher grade of CD25 expression, or a higher percentage distribution of one or more grades, may determine a higher dose of anti-CD25-ADC, or more aggressive treatment, is suitable. In some cases, a higher expression of CD25, or a higher percentage distribution of one or more grade of CD25 expression may indicate that anti-CD25-ADC should be administered with an additional therapeutic agent such as a chemotherapeutic agent.

In certain aspects, the expression of CD25 in a sample is determined by immunohistochemistry. In some cases, the sample is a formalin fixed paraffin embedded (FFPE) sample. In some cases, the sample is a frozen sample.

In some aspects, the subject has received a diagnosis of cancer. In some cases, the subject has received a diagnosis of Hodgkin's or non-Hodgkin's lymphoma. In other cases, the subject has received a diagnosis of solid cancers, where there is a proportion of CD25 expressing non-tumour cells, such as infiltrating T-cells.

In certain aspects, an anti-CD25-ADC is administered to a subject who has been determined to be suitable for treatment, or has been selected for treatment using a method described herein. In some aspects, an anti-CD25-ADC is administered for the treatment of cancer, wherein the patient has been determined to be suitable for treatment, or has been selected for treatment, using a method disclosed herein. The anti-CD25-ADC may be one disclosed in WO2014/057119. In certain aspects, the anti-CD25-ADC is ADCT-301.

In some aspects, use of an anti-CD25-ADC for the manufacture of a medicament for the treatment of cancer in a subject is provided. In these aspects, the subject has been determined to be suitable for treatment, or has been selected for treatment, with anti-CD25-ADC. The anti-CD25-ADC may be ADCT-301.

Also described is the use of an anti-CD25 antibody for determining the suitability of a subject for treatment with an anti-CD25-ADC. The antibody may be antibody 4C9. 4C9 antibodies are obtainable from Cell Marque antibodies by Ventana Medical Systems, Inc, Catalogue Number 760-4439. The antibody may be used in an immunohistochemical method. The antibody may be incubated with a formalin fixed paraffin embedded sample from the subject. The sample may prepared with, and the antibody may be incubated with the sample, using an automated slide staining system, such as a Ventana™ BenchMark ULTRA™ platform, Ventana Discovery™, Dako Omnis™, Dako AutostainerLink48™ or Leica™ BOND RX™, Leica™ BOND-III™ or Leica™ BOND MAX™. The antibody may be labelled.

Another aspect disclosed herein is a method for determining that a subject is suitable for treatment with an anti-CD25-ADC, the method comprising
performing an immunohistochemical analysis of a sample from a subject, the sample having been fixed and incubated with an anti-CD25 antibody; and
determining the expression of CD25 in the sample;
   wherein the subject is determined to be suitable for treatment with an anti-CD25-ADC based on the expression of CD25 in the sample.

### DETAILED DESCRIPTION

An immunohistochemistry (IHC) test for determination of CD25 protein expression in cancer tissue is disclosed, to select subjects for treatment with ADCT-301, an antibody drug conjugate composed of a human antibody against human CD25 attached to a pyrrolobenzodiazepine (PBD) warhead via a cleavable linker. This test may be a companion diagnostic test for ADCT-301, such as a laboratory development test (LDT) or in vitro diagnostic test (IVD). The test may be a commercially distributed in vitro diagnostic (IVD) companion diagnostic test kit. The CD25-PBD-ADC product label may require determination of CD25 expression by this test

The test may be a semi-quantitative immunohistochemical assay for the determination of CD25 protein expression in formalin-fixed, paraffin-embedded, cancer tissue. Tissue from subjects with tumors likely to be CD25 positive, such as Hodgkin's lymphoma, and Non-Hodgkin's Lymphomas (NGL) such as; cutaneous T cell lymphoma (CTCL), peripheral T cell lymphoma (PTCL) or refractory diffuse large B cell lymphoma (DLBCL) will be tested, to aid in identifying those subjects expected to benefit from treatment with ADCT-301. Patients with solid tumors containing high levels of CD25 infiltrating T-cells may be eligible for treatment with ADCT-301.

Antibody drug conjugates (ADCs) provide a real opportunity for targeted delivery of highly potent chemotherapeutic agents to tumor targets. In non-clinical safety studies however, the specific targeting aspect is often lost/diminished due to the lack of target expression (either expression level, location or different pharmacology) and/or lack of/reduced binding of the ADC to the non-clinical species target. As such non-clinical safety studies often highlight potential off-target toxicities of ADCs (which in the therapeutic clinical setting may be offset by target expression at the tumor site), demonstrating the need for selection of subjects with target expression to provide the best risk-benefit profile. Based on mechanistic and toxicology data, it is expected that CD25-negative subjects will have no, or minimal, response to ADCT-301 and could potentially be exposed to unreasonable risk.

Whilst false negative results could withhold a subject from a treatment he/she could hope to benefit from, false positive results could lead to treatment of a subject with ADCT-301 with little chance of therapeutic benefit. Thus, robust and accurate identification of a responder population (i.e. subjects with CD25 positive cells in the tumor) to enhance the benefit-risk relationship of ADCT-301 is important.

There is no FDA approved in vitro diagnostic for the determination of CD25 expression to aid in identifying subjects eligible for treatment with CD25 targeting agents in the malignancies to be studied during clinical analysis of ADCT-301.

Quest Diagnostics offers a CD25 immunohistochemistry Laboratory Developed Test (LDT) used to determine eligibility for ONTAK® (denileukin diftitox) treatment in subjects with persistent or recurrent cutaneous T-cell lymphoma. This test was developed and its performance characteristics have been determined by Quest Diagnostics Nichols Institute, but it has not been cleared or approved by FDA. In addition, cut-off and quality of testing in CTCL seem to be sub-optimal and seem to have been chosen quite arbitrarily (Talpur, Jones et al. 2006, Prince, Martin et al. 2013).

Ventana™ and many others offer CD25 immunohistochemistry kits that are approved IVDs, however with a different intended use, (the Ventana CD25 IVD is used for the determination of CD25 expression on mast cell aggregates in bone marrow is a major diagnostic criteria for systemic mastocytosis for instance).

The test may be performed as an LDT in a hospital or central laboratory with Clinical Laboratory Improvement Amendments (CLIA) accreditation. The test may be an in vitro diagnostic (IVD) test, such as a commercially distributed in vitro diagnostic test kit. For subject selection for ONTAK in CTCL the cut-off was chosen as at least 20% lymphocytes expressing CD25 but this cut-of seems to lack thorough clinical validation and appears to be sub-optimal (Talpur, Jones et al., 2006; Prince, Martin, et al., 2013). On the other hand, a cut-off of at least 10% of the lymphocytes expressing CD25 was applied in a clinical trial evaluating a radiotherapy using anti CD25 antibodies (Waldmann, White et al 1995).

In addition, a bystander effect, as discussed by Kovtun et al (Kovtun, Audette et al 2006) is also possible with ADCT-301, with the released pyrrolobenzodiazepine (PBD) dimer having an anti-tumor effect on adjacent cells. As such, targeting CD25 positive tumor infiltrating cells may well be clinically efficacious, even when the tumor cells are CD25 negative. Therefore, because of the high frequency of CD25 staining of infiltrating cells in Lymphomas, subjects with CD25 positive infiltrating cells will be eligible even if their tumor cells are CD25 negative.

CD25 determination, usually by flow cytometry is already performed as a diagnostic tool in Adult T cell leukemia (ATL) (Dasanu 2011) and Hairy Cell Leukemia (HCL) (Shao, Calvo et al., 2013).

Patient Selection

Methods disclosed herein relate to the selection or classification of subjects suitable for treatment with an Antibody Drug Conjugate. In certain aspects, the methods are useful for selecting subjects suitable for treatment with ADCT-301.

As used herein, subjects who are considered suitable for treatment are those subjects who are expected to benefit from, or respond to, the treatment. Subjects may have, or be suspected of having, or be at risk of having cancer. Subjects may have received a diagnosis of cancer. In particular, subjects may have, or be suspected of having, or be at risk of having, lymphoma. In some cases, subjects may have, or be suspected of having, or be at risk of having, a solid cancer that has CD25+ tumour associated non-tumor cells, such as CD25+ infiltrating T-cells.

In some aspects, subjects are selected on the basis of the amount or pattern of expression of a target. In certain aspects, the target is CD25. In some aspects, the selection is based on CD25 expression at the cell surface.

In some cases, expression of the target in a particular tissue of interest is determined. For example, in a sample of lymphoid tissue or tumor tissue. In some cases, systemic expression of the target is determined. For example, in a sample of circulating fluid such as blood, plasma, serum or lymph.

In some aspects, the subject is selected as suitable for treatment due to the presence of target expression in a sample. In those cases, subjects without target expression may be considered not suitable for treatment.

In other aspects, the level of target expression is used to select a subject as suitable for treatment. Where the level of expression of the target is above a threshold level, the subject is determined to be suitable for treatment.

In some aspects, the presence of CD25 in cells in the sample indicates that the subject is suitable for treatment with ADCT-301. In other aspects, the amount of CD25 expression must be above a threshold level to indicate that the subject is suitable for treatment with ADCT-301. In some aspects, the observation that CD25 localization is altered in the sample as compared to a control indicates that the subject is suitable for treatment.

In some aspects, a subject is indicated as suitable for treatment if cells obtained from lymph node or extra nodal sites react with anti-CD25 as determined by IHC.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express CD25. In some aspects disclosed herein, a patient is determined to be suitable for treatment if at least at least 10% of the cells in the sample express CD25. In some aspects, the sample contains lymphocytes, and the percentage refers to the number of lymphocytes and/or Reed-Sternberg cells in the sample that express CD25. In cases where the cancer is a solid cancer associated with non-cancerous cells, such as infiltrating T cells, the patient may be determined to be suitable for treatment based on the CD25 expression of the non-cancerous cells.

In certain aspects, a sample is appointed a grade, based on the level of expression of the target. The grading may be made on the basis of all of the cells in the sample, or on the basis of a subset of such cells, for example, expression in the lymphocytes and/or Reed-Sternberg cells only. A sample from a subject may be graded based on the following classifications:

Grade 1, or minimal, staining are assigned where a granular to smooth, often cytoplasmic with minimal membranous localization, staining distribution was observed. Optionally, staining may be confirmed via a lack of adjoining accompanying stromal/parenchymal staining.

Grade 2, or moderate, staining is assigned to samples which meet the requirements for grade 1 staining, and additionally have a clear delineation of, and localization to, the membrane of individual cells.

Grade 3, or marked, staining is assigned to samples which have a diffuse, often circumferential, dark membranous staining pattern.

In addition to grading the degree of staining as above, the sample may also be assigned a percentage of distribution. The percentage of distribution is a measure of the proportion of positive cells within each sample. Where a sample contains 10-20% of grade 2 staining cells, with the remaining population of cells having grade 1 staining, the sample is assigned grade 2 staining overall.

In certain aspects, a subject may be selected as suitable for treatment if 5%, 10%, 15%, 20%, 25%, 30%, 35%, 40%, 45%, 50%, 55%, 60%, 65%, 70%, 75%, 80%, 85%, 90% or more of the cells in the sample display grade 1 staining. In some cases, a subject is selected for treatment if 10% or more, 20% or more, or 30% or more, or 40% or more, or 50% or more, of the cells in the sample display grade 1 staining.

In certain aspects, a subject may be selected as suitable for treatment if 5%, 10%, 15%, 20%, 25%, 30%, 35%, 40%, 45%, 50%, 55%, 60%, 65%, 70%, 75%, 80%, 85%, 90% or more of the cells in the sample display grade 2 staining. In some cases, a subject is selected for treatment if 10% or more, 20% or more, or 30% or more, or 40% or more, 50% or more or 60% or more of the cells in the sample display grade 2 staining.

In certain aspects, a subject may be selected as suitable for treatment if 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50% or more of the cells in the sample display grade 3 staining. In some cases, a subject is selected for treatment if 1% or more, 2% or more, or 3% or more, or 4% or more, 5% or more, 6% or more, 7% or more, 8% or more, 9% or more, 10% or more, 12% or more, 15% or more or 20% or more, of the cells in the sample display grade 3 staining.

In some cases, a subject may be selected as suitable for treatment based on a combination of percentage distributions. That is, a subject may be determined as suitable for treatment if they have above a threshold of grade 1 staining and above a threshold of grade 2 staining. Where a combination of percentage distributions is used, the threshold values may be lower than where only a single percentage distribution is used.

Where the sample is a fluid sample, the method may involve detection of soluble CD25. In these cases, a subject may be determined to be suitable for treatment if the level of CD25 in the sample is elevated or overexpressed as compared to a control sample. In certain aspects, the subject is determined to be suitable for treatment if the level of CD25 is determined to be greater than 500 pg/ml, greater than 1000 pg/ml, greater than 1500 pg/ml, greater than 2000 pg/ml, greater than 2500 pg/ml, greater than 3000 pg/ml, greater than 3500 pg/ml, greater than 4000 pg/ml, greater than 4500 pg/ml or greater than 5000 pg/ml.

Samples

The sample may comprise or may be derived from: a quantity of blood; a quantity of serum derived from the individual's blood which may comprise the fluid portion of the blood obtained after removal of the fibrin clot and blood cells; a quantity of pancreatic juice; a tissue sample or biopsy; or cells isolated from said individual.

A sample may be taken from any tissue or bodily fluid. In certain aspects, the sample may include or may be derived from a tissue sample, biopsy, resection or isolated cells from said individual.

In certain aspects, the sample is a tissue sample. The sample may be a sample of tumor tissue, such as cancerous tumor tissue. The sample may have been obtained by a tumor biopsy. In some aspects, the sample is a lymphoid tissue sample, such as a lymphoid lesion sample or lymph node biopsy. In some cases, the sample is a skin biopsy.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a subject's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of a CD25 polypeptide or nucleic acid in the blood, in white blood cells, peripheral blood mononuclear cells, granulocytes and/or red blood cells.

The sample may be fresh or archival. For example, archival tissue may be from the first diagnosis of a subject, or a biopsy at a relapse. In certain aspects, the sample is a fresh biopsy.

In some aspects disclosed herein, a subject has, or is suspected as having, or has been identified as being at risk of, cancer. In some aspects disclosed herein, the subject has already received a diagnosis of cancer. The subject may have received a diagnosis of (classical) Hodgkins lymphoma (including nodular sclerosing, lymphocyte predominant, lymphocyte, or mixed cellularity type, or where the type is unspecified), diffuse large B cell lymphoma (DLBCL) or peripheral T cell lymphoma (PTCL) (including the subtypes ALCL: anaplastic large cell lymphoma or AITL: angioimmunoblastic T cell lymphoma). In some cases, the subject has received a diagnosis of nodular sclerosing or mixed cellularlity classical Hodgkins lymphoma, diffuse large B cell lymphoma, or angioimmunoblastic T cell lymphoma.

In some cases, the subject has received a diagnosis of (classical) Hodgkins lymphoma (mixed cellularity type), or non-Hodgkins lymphoma (including B-cell chronic lymphatic leukemia, diffuse large B-cell lymphoma (DLBCL), follicular lymphoma, (FL), Mantle Cell lymphoma (MCL), chronic lymphatic lymphoma (CLL) and leukemias such as Hairy cell leukemia (HCL), Hairy cell leukemia variant (HCL-v), Acute Myeloid Leukaemia (AML), Acute Lymphoblastic Leukaemia (ALL) such as Philadelphia chromosome-positive ALL (Ph+ALL) or Philadelphia chromosome-negative ALL (Ph−ALL) [Fielding A., Haematologica. 2010 January; 95(1): 8-12], small cell lymphocytic lymphoma, adult T-cell leukemia/lymphoma, and anaplastic large cell lymphoma.

In some cases, the subject has received a diagnosis of cutaneous T-cell lymphoma, mycosis fungoides, Sezary syndrome, systemic mastocytosis, B-cell lymphoma, non-hematopoietic tumors, peripheral T cell lymphoma and histiocytic proliferation.

In some cases, the subject has received a diagnosis of a solid cancer containing CD25+ expressing infiltrating T-cells.

Controls

In some aspects, target expression in the subject is compared to target expression in a control. Controls are useful to support the validity of staining, and to identify experimental artefacts.

In some cases, the control may be a reference sample or reference dataset. The reference may be a sample that has been previously obtained from a subject with a known degree of suitability. The reference may be a dataset obtained from analyzing a reference sample.

Controls may be positive controls in which the target molecule is known to be present, or expressed at high level, or negative controls in which the target molecule is known to be absent or expressed at low level.

Controls may be samples of tissue that are from subjects who are known to benefit from the treatment. The tissue may be of the same type as the sample being tested. For example, a sample of tumor tissue from a subject may be compared to a control sample of tumor tissue from a subject who is known to be suitable for the treatment, such as a subject who has previously responded to the treatment.

In some cases the control may be a sample obtained from the same subject as the test sample, but from a tissue known to be healthy. Thus, a sample of cancerous tissue from a subject may be compared to a non-cancerous tissue sample.

In some cases, the control is a cell culture sample.

In some cases, a test sample is analyzed prior to incubation with an antibody to determine the level of background staining inherent to that sample.

In some cases an isotype control is used. Isotype controls use an antibody of the same class as the target specific antibody, but are not immunoreactive with the sample. Such controls are useful for distinguishing non-specific interactions of the target specific antibody.

The methods may include hematopathologist interpretation of morphology and immunohistochemistry, to ensure accurate interpretation of test results. The method may involve confirmation that the pattern of expression correlates with the expected pattern. For example, where the amount of CD25 expression is analyzed, the method may involve confirmation that in the test sample the expression is observed as membrane staining, with a cytoplasmic component. The method may involve confirmation that the ratio of target signal to noise is above a threshold level, thereby allowing clear discrimination between specific and non-specific background signals.

CD25

The type I transmembrane protein CD25 is present on activated T- and B-cells, some thymocytes, myeloid precursors, and oligodendrocytes. On activated T-cells, it forms heterodimers with the beta- and gamma subunits (CD122 and CD132), thus comprising the high-affinity receptor for IL-2. This ligand represents a survival factor for activated T-cells, as removal of IL-2 leads to immediate death of these cells.

In case of B-cells, CD25 is physiologically expressed in early developmental stages of late pro-B and pre-B cells. Malignancies arising from this stage of B-cell differentiation may thus also express CD25. Mast cell lesions are also positive for CD25 which is thus considered as a key diagnostic criterion for determination of systemic mastocytosis. In Hodgkin lymphomas, CD25 is reported to be not expressed in Hodgkin-/Reed-Sternberg cells in nodular lymphocyte predominance Hodgkin lymphoma (NLPHL), whereas the same cell type expresses CD25 at varying levels in classical Hodgkin' lymphomas of mixed cellularity type. The general expression levels are reported to be lower than in tumor infiltrating lymphocytes (TILs), which may result in problems demonstrating CD25 tumor cells in these cases (Levi et al., Merz et al, 1995).

Expression of the target antigen has also been reported for several B- and T-cell-derived subtypes of non-Hodgkin-lymphomas, i.e. B-cell chronic lymphatic leukemia, hairy cell leukemia, small cell lymphocytic lymphoma/chronic lymphocytic leukemia as well as adult T-cell leukemia/lymphoma and anaplastic large cell lymphoma.

CD25 may be localised to the membrane, with some expression observed in the cytoplasm. Soluble CD25 may also be observed outside of cells, such as in serum.

As used herein, “binds CD25” is used to mean the antibody binds CD25 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds CD25 with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The antibodies of the disclosure can bind CD25 with a high affinity. For example, in some embodiments the antibody can bind CD25 with a K_{D} equal to or less than about 10⁻⁶ M, such as equal to or less than one of 1×10⁻⁶, 10⁻⁷, 10⁻⁸, 10⁻⁹, 10⁻¹⁰, 10⁻¹¹, 10⁻¹², 10-¹³ or 10⁻¹⁴.

In some embodiments, CD25 polypeptide corresponds to Genbank accession no. NP_000408, version no. NP_000408.1 GI:4557667, record update date: Sep. 9, 2012 04:59 PM. In one embodiment, the nucleic acid encoding CD25 polypeptide corresponds to Genbank accession no. NM_000417, version no. NM 000417.2 GI:269973860, record update date: Sep. 9, 2012 04:59 PM. In some embodiments, CD25 polypeptide corresponds to Uniprot/Swiss-Prot accession No. P01589.

Antibody Drug Conjugates (ADCs)

The present disclosure relates to diagnostic tests that may be useful for determining the eligibility of a subject to receive an ADC based therapy. The disclosure particularly relates treatment with an ADC disclosed in WO2014/057119.

The ADC can deliver a drug to a target location. The target location is preferably a proliferative cell population. The antibody is an antibody for an antigen present on a proliferative cell population. In one aspect the antigen is absent or present at a reduced level in a non-proliferative cell population compared to the amount of antigen present in the proliferative cell population, for example a tumour cell population.

The ADC may comprise a linker which may be cleaved so as to release the drug at the target location. The drug may be a compound selected from RelA, RelB, RelC, RelD or RelE. Thus, the conjugate may be used to selectively provide a compound RelA, RelB, Rel C, RelD or RelE to the target location.

The linker may be cleaved by an enzyme present at the target location.

As used herein, the term “CD25-ADC” refers to an ADC in which the antibody component is an anti-CD25 antibody. The term “PBD-ADC” refers to an ADC in which the drug component is a pyrrolobenzodiazepine (PBD) warhead. The term “anti-CD25-ADC refers to an ADC in which the antibody component is an anti-CD25 antibody, and the drug component is a PBD warhead.

The ADC may comprise a conjugate of formula L-(D^{L})ₚ, where D^{L} is of formula I or II:

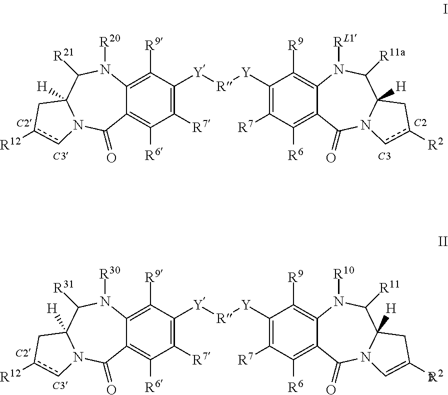

wherein:

L is an antibody (Ab) which is an antibody that binds to CD25, the antibody comprising:
a VH domain comprising a VH CDR1 with the amino acid sequence of SEQ ID NO.3, a VH CDR2 with the amino acid sequence of SEQ ID NO.4, and a VH CDR3 with the amino acid sequence of SEQ ID NO.5;
when there is a double bond present between C2′ and C3′, R¹² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

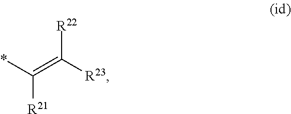

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

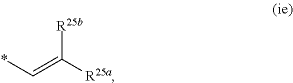

wherein one of R^{25a} and R^{25b} is H and the other is selected from: phenyl, which phenyl is optionally substituted by a group selected from halo, methyl, methoxy; pyridyl; and thiophenyl; and

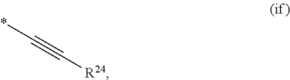

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2′ and C3′,

R¹² is

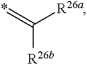

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;

R⁶ and R⁹ are independently selected from H, R, OH, OR, SH, SR, NH₂, NHR, NRR′, nitro, Me3Sn and halo;

where R and R′ are independently selected from optionally substituted C₁₋₁₂ alkyl, C₃₋₂₀ heterocyclyl and C₅₋₂₀ aryl groups;

R⁷ is selected from H, R, OH, OR, SH, SR, NH₂, NHR, NHRR′, nitro, Me₃Sn and halo;

R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;

Y and Y′ are selected from O, S, or NH;

R^{6′}, R^{7′}, R^{9′} are selected from the same groups as R⁶, R⁷ and R⁹ respectively;

[Formula I]

R^{L1′} is a linker for connection to the antibody (Ab);

R^{11a} is selected from OH, OR^{A}, where R^{A} is C₁₋₄ alkyl, and SO_{z}M, where z is 2 or 3 and M is a monovalent pharmaceutically acceptable cation;

R²⁰ and R²¹ either together form a double bond between the nitrogen and carbon atoms to which they are bound or;

R²⁰ is selected from H and R^{C}, where R^{C} is a capping group;

R²¹ is selected from OH, OR^{A} and SO_{z}M;

when there is a double bond present between C2 and C3, R² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

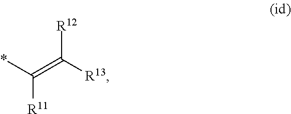

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

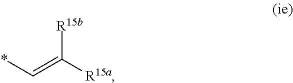

wherein one of R^{15a} and R^{15b} is H and the other is selected from: phenyl, which phenyl is optionally substituted by a group selected from halo, methyl, methoxy; pyridyl; and thiophenyl; and

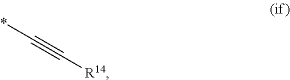

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2 and C3,

R² is

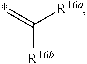

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

[Formula II]

R²² is of formula IIIa, formula IIIb or formula IIIc:

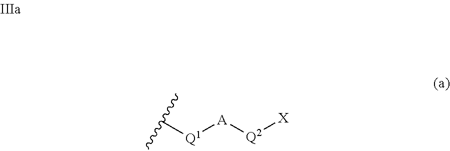

where A is a C₅₋₇ aryl group, and either

(i) Q¹ is a single bond, and Q² is selected from a single bond and —Z—(CH₂)ₙ—, where Z is selected from a single bond, O, S and NH and n is from 1 to 3; or

(ii) Q¹ is —CH═CH—, and Q² is a single bond;

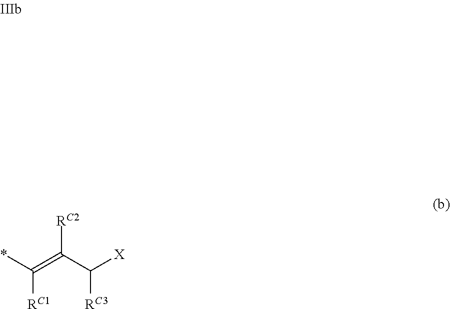

where;

R^{C1}, R^{C2} and R^{C3} are independently selected from H and unsubstituted C₁₋₂ alkyl;

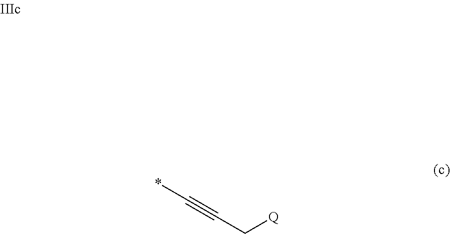

where Q is selected from O—R^{L2′}, S—R^{L2′} and NR^{N}—R^{L2′}, and R^{N} is selected from H, methyl and ethyl

X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′},

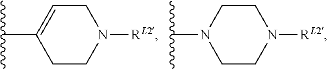

NR^{N}R^{L2′}, wherein R^{N} is selected from the group comprising H and C₁₋₄ alkyl;

R^{L2′} is a linker for connection to the antibody (Ab);

R¹⁰ and R¹¹ either together form a double bond between the nitrogen and carbon atoms to which they are bound or;

R¹⁰ is H and R¹¹ is selected from OH, OR^{A} and SO_{z}M;

R³⁰ and R³¹ either together form a double bond between the nitrogen and carbon atoms to which they are bound or;

R³⁰ is H and R³¹ is selected from OH, OR^{A} and SO_{z}M.

In some embodiments L-R^{L1′} or L-R^{L2′} is a group:

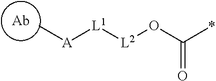

where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.

In some of these embodiments, L¹ is enzyme cleavable.

It has previously been shown that such ADCs are useful in the treatment of CD25 expressing cancers (see, for example, WO2014/057119, which is incorporated by reference herein in its entirety).

The term anti-CD25-ADC may include any embodiment described in WO 2014/057119. In particular, in some embodiments the ADC may have the chemical structure:

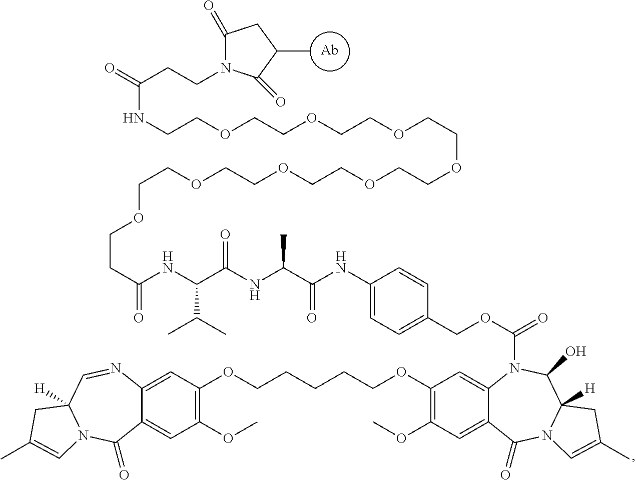

where the Ab is a CD25 antibody, and the DAR is between 1 and 8.

In some aspects the antibody component of the anti-CD25-ADC is an antibody comprising: a VH domain comprising a VH CDR1 with the amino acid sequence of SEQ ID NO.3, a VH CDR2 with the amino acid sequence of SEQ ID NO.4, and a VH CDR3 with the amino acid sequence of SEQ ID NO.5. In some embodiments the antibody comprises a VH domain having the sequence according to SEQ ID NO. 1.

The antibody may further comprise: a VL domain comprising a VL CDR1 with the amino acid sequence of SEQ ID NO.6, a VL CDR2 with the amino acid sequence of SEQ ID NO.7, and a VL CDR3 with the amino acid sequence of SEQ ID NO.8. In some embodiments the antibody further comprises a VL domain having the sequence according to SEQ ID NO. 2.

In some embodiments the antibody comprises a VH domain and a VL domain, the VH and VL domains having the sequences of SEQ ID NO. 1 paired with SEQ ID NO. 2.

The VH and VL domain(s) may pair so as to form an antibody antigen binding site that binds CD25.

In some embodiments the antibody is an intact antibody comprising a VH domain and a VL domain, the VH and VL domains having sequences of SEQ ID NO. 1 and SEQ ID NO. 2.

In some embodiments the antibody is a fully human monoclonal IgG1 antibody, preferably IgG1,k.

In some embodiments the antibody is the AB12 antibody described in WO 2004/045512 (Genmab A/S).

In an aspect the antibody is an antibody as described herein which has been modified (or further modified) as described below. In some embodiments the antibody is a humanized, deimmunised or resurfaced version of an antibody disclosed herein.

ADCT-301

ADCT-301 is an antibody drug conjugate composed of a human antibody against human CD25 attached to a pyrrolobenzodiazepine (PBD) warhead via a cleavable linker. The mechanism of action of ADCT-301 depends on CD25 binding. The CD25 specific antibody targets the antibody drug conjugate (ADC) to cells expressing CD25. Upon binding, the ADC internalizes and is transported to the lysosome, where the protease sensitive linker is cleaved and free PBD dimer is released inside the target cell. The released PBD dimer inhibits transcription in a sequence-selective manner, due either to direct inhibition of RNA polymerase or inhibition of the interaction of associated transcription factors. The PBD dimer produces covalent crosslinks that do not distort the DNA double helix and which are not recognized by nucleotide excision repair factors, allowing for a longer effective period (Hartley 2011).

It has the chemical structure:

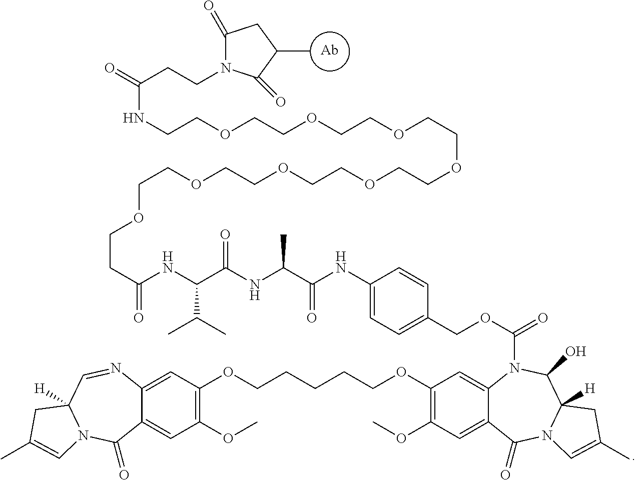

Ab represents Antibody AB12 (fully human monoclonal IgG1, K antibody with the VH and VL sequences Seq 1 and Seq 2, respectively, also known as HuMax-TAC). It is synthesised as described in WO 2014/057119 (Conj AB12-E) and has a DAR (Drug to Antibody Ratio) of 2.3.

Methods

Methods according to the present disclosure may be performed in vitro or ex vivo. The term “in vitro” is intended to encompass experiments with materials, biological substances, cells and/or tissues in laboratory conditions or in culture. “Ex vivo” refers to something present or taking place outside an organism, e.g. outside the human or animal body, which may be on tissue (e.g. whole organs) or cells taken from the organism.

The methods disclosed herein relate to the determination of protein expression. Protein expression can be measured by quantifying the amount of protein in a cell, tissue or sample, or by observing the localization of the protein within cells and tissues.

In some cases, immunoassays are used to detect the target (CD25) in a sample from the subject. Immunoassays use antibodies with specific affinity for the target molecule in conjunction with a detectable molecule. In some cases, the antibody is conjugated to the detectable molecule. The detectable molecule may be referred to as a label. The detectable molecule produces a detectable signal when the antibody is bound to the target molecule. The detectable signal may be a quantifiable signal. In some cases, an aptamer is used instead of, or together with, the antibody. Immunoassays include immunohistochemistry, ELISA, immunoblotting and flow cytometry. In certain aspects described herein, the assay is an immunohistochemistry assay. Such assays commonly use antibodies, although other target specific molecules such as aptamers or other ligands may be used.

The method may be approved for use by a regulatory agency. The method may be an FDA approved method.

Immunohistochemistry

Immunohistochemistry (IHC) is broadly used and well established as a diagnostic test methodology particularly in oncology indications and provides highly accurate results if used under standardized conditions (Demidova, Barinov et al., 2014).

IHC refers to the process of detecting targets in cells of a tissue section by exploiting the principle of antibodies binding specifically to the target in biological tissues. IHC is widely used in the diagnosis of abnormal cells, such as those found in cancerous tumours. Visualizing an antibody-target interaction can be accomplished in a number of ways. Commonly, an antibody is conjugated to label. Alternatively, the antibody is detected by a secondary antibody, which is itself labelled. Detection of the label is thus indicative of the presence of target. IHC can be used to determine the cellular localization of a target and the amount of target present. IHC may be qualitative or semi-quantitative.

Immunohistochemistry methods are known in the art and are suitable for use as described herein. IHC methods commonly involve the fixation of a sample so that the sample is preserved from degradation. In certain aspects, a sample is formalin fixed and paraffin embedded (FFPE). In other aspects, IHC is performed on frozen samples. Prepared samples may be sectioned prior to analysis.

The sample may undergo pre-treatment, such as with Ventana CC1 (Cell Conditioning 1) solution. Where the sample is a FFPE sample, the method may involve deparaffinisation of the sample.

Prepared samples are incubated with an antibody that is specific to the target. The samples may be incubated with an anti-CD25 antibody. The conditions and duration of incubation will depend on the particular antibody used. In some cases, the sample is incubated for between 10 minutes and 60 minutes, between 20 minutes and 45 minutes, or between 25 minutes and 35 minutes. In some cases, the sample may be incubated with the antibody for around 30 minutes, such as for 32 minutes. Incubation may occur at room temperature, or between about 20° C. and 50° C., between 30° C. and 40° C., or around 35° C., such as 37° C. Preferably the sample is incubated with the antibody for 32 minutes at 37° C.

The samples may additionally be counter-stained to facilitate analysis. For example, the sample may be stained with haematoxylin and eosin (H&E) stained.

The methods disclosed herein may be performed manually or automatically. Preferably, the methods are at least partially automated. For example, slide staining steps may be automated. Slide staining may be performed using a Ventana™ BenchMark ULTRA™. Alternatively, slide staining may be performed using a Ventana™ BenchMark XT™, Ventana™ BenchMark GX™, Dako Omnis™, Dako AutostainerLink48™, Leica™ BOND RX™, Leica™ BOND-III™ or Leica™ BOND MAX™

In some cases, the Quest Diagnostics CD25 IHC with interpretation LDT will be used to select subjects with treatment.

Following incubation of the sample with the labelled antibody, they may be analyzed using a microscope.

ELISA

In some cases, the target may be detected by ELISA (enzyme-linked immunosorbent assay). Target molecules from a sample are attached to a surface and detected using a specific antibody. The target may be attached to the surface non-specifically (via adsorption to the surface) or specifically (using a specific capture agent such as an antibody). ELISA may be used to quantify target in a sample. ELISA is particularly suited to the analysis of liquid samples, such as serum, urine or saliva.

Immunoblotting

In some aspects, the target is detected by immunoblotting, or western blotting. In such methods, proteins in a sample are separated based on their electrical charge or size. They may be separated by an electrophoresis based method. The separated proteins are transferred to a membrane, where they are stained with an antibody that is specific to the target. The antibody is then detected, either directly by virtue of the antibody being conjugated to a detectable label, or indirectly, by adding a labelled secondary antibody.

Flow Cytometry

Flow cytometry based biomarker detection may be used to detect cells expressing a biomarker of interest, such as CD25. Cells from the sample are suspended in a stream of fluid and directed past an electronic detection apparatus. The cells may be labelled with an antibody that is specific to the biomarker of interest. In particular, the cells may be labelled with a fluorescent antibody. Cells that express the biomarker of interest may be detected and quantified, based on the fluorescent signal from the label.

A type of flow cytometry useful in the methods disclosed herein is Fluorescence Activated Cell Sorting (FACS). Using FACS, cells may be separated into two or more vessels, based on the presence or absence of the fluorescent label on the cell.

Hybridization

Certain aspects herein relate to the detection of a nucleic acid of interest, such as a CD25 nucleic acid. The nucleic acid may be a genomic nucleic acid or a transcribed nucleic acid, such as a mRNA. The method may involve the generation of cDNA from a mRNA of interest.

Suitable methods for the detection of nucleic acids include a hybridization step, in which a nucleic acid of interest is complementary to, and binds to, a nucleic acid molecule with a known sequence. The nucleic acid molecule with a known sequence may be a probe or primer, and may be synthetic. It may be labelled, such as with a radioactive moiety or a colourimetric moiety. Nucleic acid detection methods may be qualitative or quantitative. Such methods may also be used to detect the location of a nucleic acid of interest within a cell, tissue or organism.

Methods for the detection of nucleic acid include PCR based methods, such as rtPCR and qPCR. Other methods include northern and Southern blotting. Such methods involve separation of fragments, such as by electrophoresis, and subsequent detection of nucleic acid by probe hybridization.

Further methods include in situ hybridization methods, such as Fluorescent in situ hybridization (FISH). FISH uses fluorescent probes that bind only those parts of the chromosome with which they show a high degree of sequence complementarity. FISH may also be used to detect RNA targets, such as mRNA. FISH may be used to detect nucleic acid in cells, circulating tumor cells and tissue samples.

Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind CD25 (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Anti-CD25 antibodies are known in the art and are useful in the methods disclosed herein. These include antibodies 4C9 (obtainable from Ventana Medical Systems, Inc.). Other suitable antibodies include antibody AB12 described in WO 2004/045512 (Genmab A/S), IL2R.1 (obtainable from Life Technologies, catalogue number MA5-12680) and RFT5 (described in U.S. Pat. No. 6,383,487). Other suitable antibodies include B489 (143-13) (obtainable from Life Technologies, catalogue number MA1-91221), SP176 (obtainable from Novus, catalogue number NBP2-21755), 1B5D12 (obtainable from Novus, catalogue number NBP2-37349), 2R12 (obtainable from Novus, catalogue number NBP2-21755), or BC96 (obtainable from BioLegend, catalogue number V T-072) and M-A251 (obtainable from BioLegend, catalogue number IV A053). Other suitable anti-CD25 antibodies are daclizumab (Zenapax™) and basiliximab (Simulect™), both of which have been approved for clinical use.

Detection and Labelling

The antibodies useful in the methods of the present disclosure may be labelled.

The target may be directly detected. That is to say that the target is detected by an anti-target antibody that is labelled.

Alternatively, detection of the target may be indirect. That is to say that the target may be detected by the anti-target antibody, and the anti-target antibody is subsequently detected by a secondary detectable antibody. The secondary antibody is preferably labelled. Suitable secondary antibodies may be raised against the antibody isotype of the animal species in which the primary antibody has been raised. For example, the secondary antibody may be an anti-mouse antibody, capable of binding to mouse antibodies. Methods using a secondary antibody may be more sensitive than direct detection methods, due to signal amplification from multiple secondary antibodies binding the each primary antibody.

In certain aspects, the methods of the disclosure involve indirect detection of the target.

Suitable labels include enzymes such as horseradish peroxidase, alkaline phosphatase, glucose oxidase and luciferase, and colourimetric agents, including quantum dots, fluorophores and chromophores. Suitable fluorophores include FITC. The label may be a radiolabel. The label may be a nucleic acid probe, and the method involves real-time immunoquantitative PCR (iqPCR). In some aspects, the antibody is labelled with horseradish peroxidase.

A variety of detectable enzymatic substrates are available for use with enzymatically labelled antibodies. These include chromogenic substrates, such as pNNP, BCIP/NBT (5-bromo-4-chloro-3′-indolyphosphate/nitro-blue tetrazolium), TMB (tetramethybenzidine), DAB (3,3′-diaminobenzidine), OPD (ortho-phenylenediaine dihydrochloride) and ABTS (2,2′-azinobis[-ethylbenzothiazoline-6-sulfonic acid]), and chemiluminscent substrates such as an ECL (enhanced chemiluminscent) label or Acridinium ester (AE).

In Vitro Diagnostics

An aspect of the present disclosure includes in vitro diagnostic methods, and in vitro diagnostic kits for performing such methods. A kit as described herein may include one or more antibodies, such as an anti-CD25 antibody or fragment thereof. The kit may be suitable for selecting a subject for treatment with an anti-CD25-ADC.

The kit may be suitable for a point-of-care in vitro diagnostic test. It may be kit for laboratory based testing. The kit may include instructions for use, such as an instruction booklet or leaflet. The instructions may include a protocol for performing any one or more of the methods described herein. The instructions may include a protocol for performing an immunochromatographic assay. They may describe methods and suggestions for adapting the test for different types of sample. They may provide methods and suggestions for optimizing the results obtained from the test, such as minimizing the signal to noise ratio.

The kit may be suitable for performing an immunochromatographic assay. In some cases, the in vitro diagnostic test involves a lateral flow device, or “dipstick” test. In some cases, the kit includes a multiwall plate or other solid support that is pre-coated with a capture agent, such as an anti-CD25 antibody.

The kit may additionally include standards or controls. The kit may additionally include buffers, diluents or other reagents, such as stop buffer, sample preparation buffer, colour development reagents or wash buffer.

The kit may be adapted for use with dry samples, wet samples, frozen samples, fixed samples, urine samples, saliva samples, tissue samples, blood samples, or any other type of sample, including any of the sample types disclosed herein.

Therapy

The methods of the disclosure may be used to select a subject for treatment with an ADC.

The antibody-drug conjugate (ADC) compounds described herein include those with utility for anticancer activity. In particular, the compounds include an antibody conjugated, i.e. covalently attached by a linker, to a PBD drug moiety, i.e. toxin. When the drug is not conjugated to an antibody, the PBD drug has a cytotoxic effect. The biological activity of the PBD drug moiety is thus modulated by conjugation to an antibody. The antibody-drug conjugates (ADC) of the disclosure selectively deliver an effective dose of a cytotoxic agent to tumor tissue whereby greater selectivity, i.e. a lower efficacious dose, may be achieved.

Thus, in one aspect, the present disclosure provides a conjugate compound as described herein for use in therapy, wherein the method comprises selecting a subject based on expression of CD25.

In one aspect, the present disclosure provides an ADC compound with a label that specifies that the ADC is suitable for use in a subject determined to be suitable for such use by a method disclosed herein. The label may specify that the ADC is suitable for use in a subject has CD25 expression, such as CD25 overexpression. The label may specify that the subject has a particular type of cancer. The cancer may be lymphoma. The label may specify that the subject has a CD25+ lymphoma.

In a further aspect there is also provides a conjugate compound as described herein for use in the treatment of a proliferative disease. Another aspect of the present disclosure provides the use of a conjugate compound in the manufacture of a medicament for treating a proliferative disease.

One of ordinary skill in the art is readily able to determine whether or not a candidate conjugate treats a proliferative condition for any particular cell type. For example, assays which may conveniently be used to assess the activity offered by a particular compound are described in the examples below.

The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis. Cancers of particular interest include, but are not limited to, leukemias and ovarian cancers.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Disorders of particular interest include, but are not limited to, Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL) and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL) [Fielding A., Haematologica. 2010 January; 95(1): 8-12].

It is contemplated that the antibody-drug conjugates (ADC) of the present disclosure may be used to treat various diseases or disorders, e.g. characterized by the overexpression of a tumor antigen. Exemplary conditions or hyperproliferative disorders include benign or malignant tumors; leukemia, haematological, and lymphoid malignancies. Others include neuronal, glial, astrocytal, hypothalamic, glandular, macrophagal, epithelial, stromal, blastocoelic, inflammatory, angiogenic and immunologic, including autoimmune disorders and graft-versus-host disease (GVHD).

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Autoimmune diseases for which the ADC compounds may be used in treatment include rheumatologic disorders (such as, for example, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g. ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, graft-versus-host disease (GVHD), and autoimmune endocrine disorders (such as, for example, diabetic-related autoimmune diseases such as insulin-dependent diabetes mellitus (IDDM), Addison's disease, and autoimmune thyroid disease (e.g. Graves' disease and thyroiditis)). More preferred such diseases include, for example, rheumatoid arthritis, ulcerative colitis, ANCA-associated vasculitis, lupus, multiple sclerosis, Sjögren's syndrome, Graves' disease, IDDM, pernicious anemia, thyroiditis, and glomerulonephritis.

In some aspects, the subject has a proliferative disorder selected from (classical) Hodgkin lymphomas, with mixed cellularity type (Hodgkin-/Reed-Sternbert-Cells: CD25 +/−), or non-Hodgkin lymphoma, including B-cell chronic lymphatic leukemaia, diffuse large B-cell lymphoma (DLBCL), follicular lymphoma, (FL), Mantle Cell lymphoma (MCL), chronic lymphatic lymphoma (CLL) and leukemias such as Hairy cell leukemia (HCL), Hairy cell leukemia variant (HCL-v), Acute Myeloid Leukaemia (AML), Acute Lymphoblastic Leukaemia (ALL) such as Philadelphia chromosome-positive ALL (Ph+ALL) or Philadelphia chromosome-negative ALL (Ph−ALL) [Fielding A., Haematologica. 2010 January; 95(1): 8-12], small cell lymphocytic lymphoma, adult T-cell leukemia/lymphoma, or anaplastic large cell lymphoma.

Classical Hodgkins lymphoma includes the subtypes nodular sclerosing, lymphocyte predominant, lymphocyte depleted and mixed cellularity. The Hodgkins lymphoma subtype may not be defined. In certain aspects, the patients tested according to the methods here have Hodgkins lymphoma of the nodular sclerosing and mixed cellularity subtypes.

In certain aspects, the subject has diffuse large B cell lymphoma or peripheral T cell lymphoma, including the anaplastic large cell lymphoma and angioimmunoblastic T cell lymphoma subtypes.

Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. The methods may use an ADC (antibody drug conjugate). The ADC may comprise an anti-CD25 antibody. The anti-CD25 antibody may be HuMax-TAC™. The ADC may comprise a drug which is a PBD dimer. The ADC may be a anti-CD25-ADC, and in particular, ADCT-301. The ADC may be an ADC disclosed in WO2014/057119. Also provided is a method of treatment, comprising administering to a subject in need of treatment a therapeutically-effective amount of an ADC. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The treatment may involve administration of the ADC alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: Lenalidomide (REVLIMID®, Celgene), Vorinostat (ZOLINZA®, Merck), Panobinostat (FARYDAK®, Novartis), Mocetinostat (MGCD0103), Everolimus (ZORTRESS®, CERTICAN®, Novartis), Bendamustine (TREAKISYM®, RIBOMUSTIN®, LEVACT®, TREANDA®, Mundipharma International), erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo- 2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1l, calicheamicin omegal1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above. Combinations of agents may be used, such as CHP (doxorubicin, prednisone, cyclophosphamide), or CHOP (doxorubicin, prednisone, cyclophopsphamide, vincristine).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C4, Genentech), trastuzumab (HERCEPTIN®, Genentech), tositumomab (Bexxar, Corixia), MDX-060 (Medarex) and the antibody drug conjugate, gemtuzumab ozogamicin (MYLOTARG®, Wyeth).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

Dosage

It will be appreciated by one of skill in the art that appropriate dosages of the conjugate compound, and compositions comprising the conjugate compound, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of ADC is determined by the expression of CD25 observed in a sample obtained from the subject. Thus, the level or localization of CD25 expression in the sample may be indicative that a higher or lower dose of ADC is required. For example, a high expression level of CD25 may indicate that a higher dose of ADC would be suitable. In some cases, a high expression level of CD25 may indicate the need for administration of another agent in addition to the ADC. For example, administration of the ADC in conjunction with a chemotherapeutic agent. A high expression level of CD25 may indicate a more aggressive therapy.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, the active compound is administered to a human subject according to the following dosage regime: about 100 mg, 3 times daily.

In one embodiment, the active compound is administered to a human subject according to the following dosage regime: about 150 mg, 2 times daily.

In one embodiment, the active compound is administered to a human subject according to the following dosage regime: about 200 mg, 2 times daily.

However in one embodiment, the conjugate compound is administered to a human subject according to the following dosage regime: about 50 or about 75 mg, 3 or 4 times daily.

In one embodiment, the conjugate compound is administered to a human subject according to the following dosage regime: about 100 or about 125 mg, 2 times daily.

The dosage amounts described above may apply to the conjugate (including the PBD moiety and the linker to the antibody) or to the effective amount of PBD compound provided, for example the amount of compound that is releasable after cleavage of the linker.

Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus. In one preferred embodiment, the subject/patient is a human. The terms “subject”, “patient” and “individual” are used interchangeably herein.

In some aspects disclosed herein, a subject has, or is suspected as having, or has been identified as being at risk of, cancer. In some aspects disclosed herein, the subject has already received a diagnosis of cancer. In some cases, the subject has a histologically confirmed diagnosis. The subject may be undergoing, or have undergone, a therapeutic treatment for that cancer. The subject may, or may not, have previously received ADCT-301. In some cases the cancer is lymphoma, including Hodgkins or non-Hodgkins lymphoma.

### BRIEF DESCRIPTION OF THE FIGURES

Embodiments and experiments illustrating the principles of the disclosure will now be discussed with reference to the accompanying figures in which:

FIG. 1. Diffuse T Cell Lymphoma—Photograph showing minimal (grade 1) expression of CD25 within the tumor population, scattered CD25-positive inflammatory cell infiltrates.

FIG. 2. Diffuse Large B Cell Lymphoma—Photograph showing moderate (grade 2) expression of CD25 within the relatively homogenous population of cells.

FIG. 3. Hodgkin Lymphoma (Mixed): Photograph showing marked (grade 3) expression of CD25. Note the highly expressing Reed Sternberg/Hodgkin subtypes within this heterogenous population of cells.

FIG. 4. Summary table of CD25 Expression cHL: classical Hodgkin's lymphoma, subtypes: NS: nodular sclerosing, LP: lymphocyte predominant; LD=lymphocyte depleted; MC=mixed cellularity; U=unspecified; § DLBCL: Diffuse large B cell lymphoma; γPTCL: Peripheral T cell lymphoma, subtypes: ALCL: Anaplastic large cell lymphoma; AITL: angioimmunoblastic T cell lymphoma.

FIG. 5. Sequences

The disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Particular Aspects

An aspect disclosed herein is a method comprising detecting CD25 in a sample obtained from a subject and determining that the subject is suitable for treatment with an anti-CD25-ADC if CD25 is expressed in cells in the sample.

An aspect disclosed herein is a method comprising detecting CD25 in a sample obtained from a subject and selecting the subject for treatment with an anti-CD25-ADC if CD25 is expressed in cells in the sample.

Another aspect disclosed herein is a method comprising administering an anti-CD25-ADC to a subject who has been determined to be suitable for treatment according to a method of any one of the preceding claims.

Another aspect disclosed herein is an anti-CD25-ADC for use in a method of treatment of a subject determined to be suitable for such treatment.

Another aspect disclosed herein is the use of an anti-CD25-ADC in the manufacture of a medicament for the treatment of cancer, wherein the subject has been selected for treatment by a method comprising determining the level of CD25 expression in a sample from a subject.

Another aspect disclosed herein is use of an anti-CD25 antibody for determining the suitability of a subject for treatment with an anti-CD25-ADC.

Another aspect disclosed herein is a method comprising determining, in a sample obtained from a subject diagnosed with lymphoma, the level of expression of CD25 and, based on the level of CD25 determined, determining whether the subject is suitable for treatment with an anti-CD25-ADC and, if the subject is determined to be suitable for treatment with an anti-CD25-ADC, administering an anti-CD25-ADC to the subject.

Another aspect disclosed herein is a method comprising administering an anti-CD25-ADC to a subject determined to be suitable for such treatment using a method disclosed herein wherein the dosage of anti-CD25-ADC is selected based on the level of CD25 expression observed.

Another aspect disclosed herein is a method comprising
performing an immunohistochemical analysis of a sample from a subject, the sample having been fixed and incubated with an anti-CD25 antibody; and
determining the expression of CD25 in the sample;
   wherein the subject is determined to be suitable for treatment with an anti-CD25-ADC based on the expression of CD25 in the sample.

Another aspect disclosed herein is a method comprising
performing an immunohistochemical analysis of a sample from a subject, the sample having been fixed and incubated with an anti-CD25 antibody; and
determining the expression of CD25 in the sample;
selecting a patient for treatment with anti-CD25-ADC based on the expression of CD25 in the sample.

### EXAMPLES

### Example 1

### CD25 Expression Profiling on Lymphoma Tissue Microarrays Using Immunohistochemistry

The objective of this study was to define and optimise the parameters for the immunohistochemistry (IHC) staining of CD25 on formalin fixed, paraffin embedded tissue (FFPE). Once defined, these conditions were used to investigate CD25 expression on human tissue microarrays (TMA's) of lymphomas.

This study was carried out in a facility compliant with the United Kingdom Statutory Instrument 1999 No. 3106, The Good Laboratory Practice Regulations 1999, as amended by the Good Laboratory Practice (Codification Amendments Etc.) Regulations 2004 and the OECD Principles on Good Laboratory Practice (revised 1997, issued January 1998) ENV/MC/CHEM(98)17. However no claim of compliance was made for this study.

This study was conducted in accordance with the relevant Propath UK Limited standard operating procedures.

Materials and Methods

CD25 antibody was antibody clone 4C9 obtained from Cell Marque. This is a murine IgG2b antibody. Batch number 126404B.

Negative control antibody was NEG CTL Mab from Roche, a murine monoclonal antibody. Batch number C11245.

FFPE spleen from cynomolgus monkey skin was used as positive control material.

Sections of this tissue were used to obtain a target-specific signal. Although the CD25 antibody is against the human protein, specific staining of B Cells was observed in the cynomolgus monkey spleen, indicating cross reactivity of the antibody with the primate protein and suitability as a positive control for the staining method. The assay was developed using data provided by the vendor.

Human Tissue Microarrays (TMAs)

A total of five human TMA's constructed from lymphomas, other tumour samples and non-diseased tissues were purchased from a commercial vendor (amsbio, Abingdon, UK).

Information on age, gender, originating organ of tumor and pathology diagnosis was included with the TMA's as well as location within the TMA to allow localization and identification of tissue cores.

Immunohistochemical Method

An indirect IHC technique using a fully-automated method on the Ventana Discovery XT platform was validated using positive control FFPE cynomolgus monkey spleen

Method Summary:
Deparaffinization of the FFPE slides using the vendors proprietary reagent and process
Blocking of endogenous protein using a casein solution
1 hour incubation with the mouse anti-CD25 antibody or negative control antibody (positive control spleen tissue only)
Detection of the mouse anti-CD25 antibody using an anti-mouse secondary reagent, conjugated to Horseradish peroxidase (HRP)
Incubation with the substrate for HRP to form a brown precipitate at the site of antibody binding in tissue
Counterstaining with hematoxylin, development of the counterstain, dehydration and mounting in a permanent mounting medium (Pertex)

Microscopic Evaluation

Slides were reviewed under a light microscope for the determination of staining. Images of staining from the human TMA's were collected and shared with the Sponsor to allow discussion and agreement of appropriate reporting strategy.

Tissue arrays were initially assessed at low magnification to ensure integrity and uniformity of staining across samples. Once confirmed, a higher magnification of individual samples allowed an assessment of positive staining and a score to be assigned. Where samples were insufficient, either due to folding of the tissue, or in cases whereby the sample had “lifted off” the slide (denoted “IS”; insufficient sample), this was marked on the individual grid. Occasionally, the presence of a smaller artefact (“AP”; artifact present) did not preclude evaluation, but was noted accordingly. For all other samples, a pragmatic scoring system was assigned, as outlined below.

Samples were individually assessed to ensure that any given tumor population was present as a sufficient proportion of the overall core, in order to ensure a representative assessment. Areas of fibrovascular stroma, both tumor-associated and preexisting, were discounted from analysis, as were areas of tumor-associated, or resident parenchymal, necrosis. Grades of staining were assigned as follows:

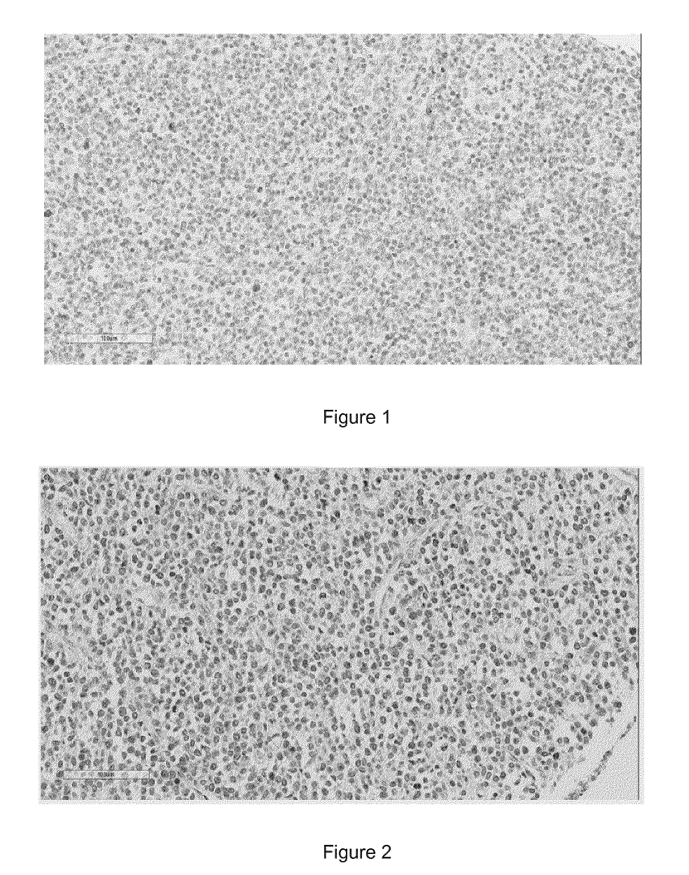

1 (minimal): A granular to smooth, often cytoplasmic with minimal membranous localization, staining distribution. Staining confirmed via a lack of adjoining/accompanying stromal/parenchymal staining (FIG. 1).

2 (moderate): As above, plus a clear delineation of, and localization to, the membrane of individual cells (FIG. 2).

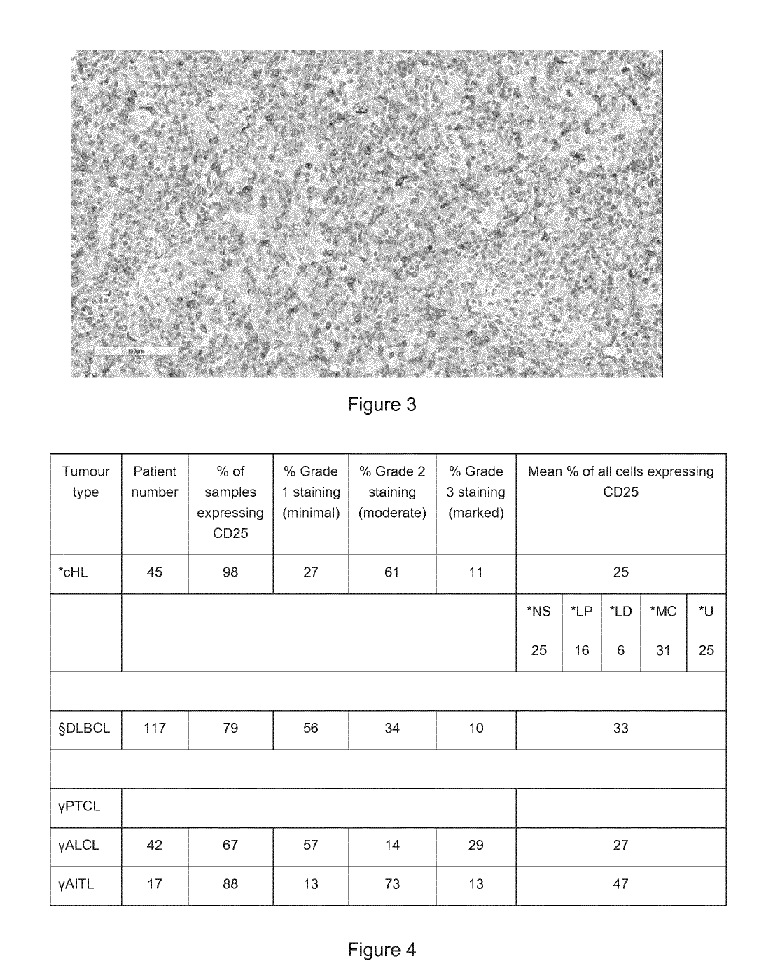

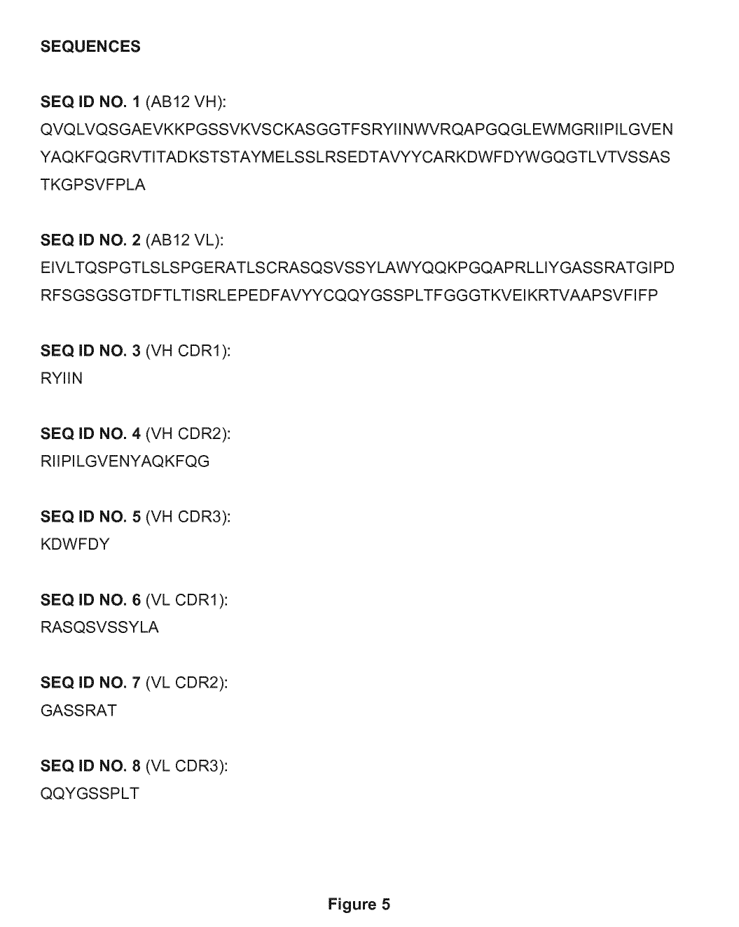

3 (marked): A diffuse, often circumferential, dark membranous staining pattern (FIG. 3).

In addition to the above grades, a “percentage of distribution” was applied to each sample. This was to give an idea as to the proportion of positive cells within each sample. If a representative sample of tumor cells, for example 10-20%, displayed grade 2 staining and the other population(s), up to a grade 1 staining, the former (higher) grade was applied to that individual sample.

In addition to a grading assignment based upon the primary neoplastic population, for some samples with a clear inflammatory component, a secondary assessment of inflammatory CD25 staining was assigned. These populations were more clearly demarcated in tumor samples with a low overall distribution/presence of positive staining as, in these cases, the scant inflammatory cell infiltrates were more easily deciphered. In samples with a greater degree of tumor-associated staining, these populations were, to an extent, “lost” amongst the positive tumor cell population(s). In addition, within some samples, the primary neoplastic population of cells was indistinguishable from secondary infiltrating lymphocytes. Further special techniques would be required in order to separate these individual sub populations.

Immunohistochemistry Results

All microarrays were of sound quality with a good distribution and uniformity of staining.

There were relatively few insufficient or absent samples. Furthermore, parenchymal/stromal (non-tumor) areas of individual samples were devoid of positive staining, imparting high confidence to the resulting dataset.

Inflammatory cell infiltrates generally comprised a low percentage of individual samples, their presence somewhat obscured in samples where there were higher percentages and intensities of resident tumor staining. In examples with a mixed and heterogeneous lymphoma population, inflammatory white blood cells were difficult to distinguish from the neoplastic population, as described above.

Overall, there was a wide range of staining intensities present within individual neoplastic populations as can be seen in Table 4.1.

Many samples were completely devoid of CD25 staining, albeit with a large proportion of these cores displaying scattered and individualized CD25-positive inflammatory cells within the sample, confirming that the specimen had been sufficiently fixed, processed and stained.

In general, lymphoma populations were consistently and uniformly stained, with the notable exception of heterogeneous and anaplastic specimens, such as Hodgkin lymphoma-derived samples. Within these notably heterogeneous cores, Reed-Sternberg (“lacunar histiocyte”) cell populations were often more intensive in their expression of the target protein. This contrasted with paler or absent staining within other infiltrating populations (Engert A et al, 1997). Mixed types predominated in their expression of the target protein, however, there were also examples of strongly positive lymphocyte-predominant and depleted subtypes.

A range of lymphoma subtypes displayed more marked staining characteristics when compared to other samples. Diffuse B/large B cell lymphomas were particularly notable in their translation of CD25, many samples displaying intense levels of expression, although this may, in part, have been a reflection of their relative frequency within the numerous microarrays. Examples of both cleaved and non-cleaved B cell lymphomas also anecdotally represented more intensively expressing cell lines.

In addition, there were several examples of strongly positive diffuse T cell lymphoma and a selection of lymphoepithelioid and mucosa-associated neoplasms with notable expression of the target protein.

Finally, and of particular note, were a selection of anaplastic (large) cell lymphomas and angioimmunoblastic T cell lymphomas which appeared over-representative in their expression of the target, when compared to no/low-expressing samples.

Summary Table of CD25 Expression

The table in FIG. 4 details a summary of CD25 expression from a few common B and T cell tumour types, showing the percentage of patient samples analyzed expressing CD25 to any degree, the proportion of staining intensity in these positive samples and a mean percentage of all cells per sample expressing CD25.

Conclusion

The analysis of a number of good-quality tissue microarrays, for the expression of the CD25, revealed this target protein to be relatively promiscuous in its expression, particularly at minimal levels, in many subtypes of malignant lymphoma. Within homogeneous subtypes, expression tended to be more uniform, becoming disparate, and often expressed to greater degrees, within heterogeneous contexts, such as Hodgkin lymphoma.

Findings within this analysis are consistent with literature-based evidence and allow for the implementation of a personalized therapy strategy (Juco et al 2003; Shao et al 2010; Barry et al 2003).

### Example 2

### Immunohistochemical Assay for Semi-Quantitative In Situ Determination of CD25 Protein Expression Levels

This study seeks to establish and validate an immunohistochemical assay for semi-quantitative in situ determination of CD25 protein expression levels in formalin-fixed and paraffin embedded human tissue specimens. Expression levels shall be determined in human lymphomas and leukemias as specified below as well as in tumor associated non-tumor cells (TANTs).

Formalin-fixed and paraffin-embedded human Hodgkin's lymphoma and non-Hodgkin's lymphoma samples (subtypes as shown below) will either be purchased from commercial providers or obtained via the Targos Pathology Network. All patient samples used within his validation project have been/will be collected with patient informed consent and/or EC approval. The Ventana/Roche anti-CD25 antibody clone 4C9 (p/n 760-4439) will be used for test establishment and validation on the Ventana benchmark Ultra platform.

H&E and IHC slides will be evaluated by a senior/board certified pathologist using standard light microscopy. IHC raw data will either be collected via versioned raw data collection forms or by validated Targos LIMS templates. Raw data analysis will include semi-quantitative detection of the percentage and intensity of stained tumor cells as well as TANTs. Statistical evaluations will be performed using Microsoft Excel 2007.

Assay establishment and validation occurs according to SOP-QA-006. In the following sections, the individual validation strategy according to pre-defined validation parameters is defined.

At least 25 samples per indication will be screened.

Raw data collection will include the percentage of stained, relevant target cells within four different intensity categories, as well as percentage of tumor infiltrating lymphocytes in relation to the tumor area.

Specificity of the assay will be verified by establishing an isotype control, competitive IHC assays with recombinant CD25 protein, staining of cell culture material expressing or not expressing target related proteins, and staining of a TMA consisting of three specimens each of all major organs. The isotype control assay will replace the original primary antibody with an Ig of the same type but not specific for any human protein, at identical titer, and will be established for use as a reagent negative control for clinical trial related patient samples.

Inter-assay repeatability will be assessed by performing three independent test runs with at least 3 different samples per indication showing different biomarker contents and concentrations (high, medium, low) in triplicate. Evaluation is performed by the same analyst. Concordance is measured separately for the percentage of stained cells of each staining category and for each relevant cellular compartment.

Inter-assay repeatability is also assessed by performing three independent test runs with three different samples per indication showing different biomarker contents and concentrations (high, medium, low) in triplicate. Evaluation is performed by the same analyst and concordance is measured separately for each relevant cellular compartment by as the percentage of stained cells for each staining category.

### REFERENCES

Levi E et al., “Detection of Interleukin-2 Receptors on Tumor Cells in Formalin-Fixed and Paraffin-Embedded Tissues” Appl Immunohistochem 199; 5: 234

Merz H et al., “ImmunoMax. A Maximized Immunohistochemical Method for the Retrieval and Enhancement of Hidden Antigens” Lab Invest 1995; 73: 149-156

Dasanu, C. A. (2011). “Newer developments in adult T-cell leukemia/lymphoma therapeutics.” Expert Opin Pharmacother 12(11): 1709-1717.

Demidova, I., A. Barinov, N. Saveloy, I. Gagarin, V. Grinevitch, D. Stroiakovaski, M. Popov, K. Laktionov, S. Gutorov, A. Smolin, Y. Olshanskaya and T. Obukhova (2014). “Immunohistochemistry, fluorescence in situ hybridization, and reverse transcription-polymerase chain reaction for the detection of anaplastic lymphoma kinase gene rearrangements in patients with non-small cell lung cancer: potential advantages and methodologic pitfalls.” Arch Pathol Lab Med 138(6): 794-802.

Hartley, J. A. (2011). “The development of pyrrolobenzodiazepines as antitumour agents.” Expert Opin Investig Drugs 20(6): 733-744.

Kovtun, Y. V., C. A. Audette, Y. Ye, H. Xie, M. F. Ruberti, S. J. Phinney, B. A. Leece, T. Chittenden, W. A. Blattler and V. S. Goldmacher (2006). “Antibody-drug conjugates designed to eradicate tumors with homogeneous and heterogeneous expression of the target antigen.” Cancer Res 66(6): 3214-3221.

Mandrekar, S. J. and D. J. Sargent (2009). “Clinical trial designs for predictive biomarker validation: theoretical considerations and practical challenges.” J Clin Oncol 27(24): 4027-4034.

Olsen, E., M. Duvic, A. Frankel, Y. Kim, A. Martin, E. Vonderheid, B. Jegasothy, G. Wood, M. Gordon, P. Heald, A. Oseroff, L. Pinter-Brown, G. Bowen, T. Kuzel, D. Fivenson, F. Foss, M. Glode, A. Molina, E. Knobler, S. Stewart, K. Cooper, S. Stevens, F. Craig, J. Reuben, P. Bacha and J. Nichols (2001). “Pivotal phase III trial of two dose levels of denileukin diftitox for the treatment of cutaneous T-cell lymphoma.” J Clin Oncol 19(2): 376-388.

Prince, H. M., A. G. Martin, E. A. Olsen, D. P. Fivenson and M. Duvic (2013). “Denileukin diftitox for the treatment of CD25 low-expression mycosis fungoides and Sezary syndrome.” Leuk Lymphoma 54(1): 69-75.

Shao, H., K. R. Calvo, M. Gronborg, P. R. Tembhare, R. J. Kreitman, M. Stetler-Stevenson and C. M. Yuan (2013). “Distinguishing hairy cell leukemia variant from hairy cell leukemia: development and validation of diagnostic criteria.” Leuk Res 37(4): 401-409.

Strauchen, J. A. and B. A. Breakstone (1987). “IL-2 receptor expression in human lymphoid lesions. Immunohistochemical study of 166 cases.” Am J Pathol 126(3): 506-512.

Talpur, R., D. M. Jones, A. J. Alencar, N. Apisarnthanarax, K. L. Herne, Y. Yang and M. Duvic (2006). “CD25 expression is correlated with histological grade and response to denileukin diftitox in cutaneous T-cell lymphoma.” J Invest Dermatol 126(3): 575-583.

Waldmann, T. A., J. D. White, J. A. Carrasquillo, J. C. Reynolds, C. H. Paik, O. A. Gansow, M. W. Brechbiel, E. S. Jaffe, T. A. Fleisher, C. K. Goldman, L. E. Top, R. Bamford, E. Zaknoen, E. Roessler, C. Kasten-Sportes, R. England, H. Litou, J. A. Johnson, T. Jackson-White, A. Manns, B. Hanchard, R. P. Junghans and D. L. Nelson (1995). “Radioimmunotherapy of interleukin-2R alpha-expressing adult T-cell leukemia with Yttrium-90-labeled anti-Tac.” Blood 86(11): 4063-4075.

1. A method comprising detecting CD25 in a sample obtained from a subject and determining that the subject is suitable for treatment with an anti-CD25-ADC if CD25 is expressed in cells in the sample.

2. A method comprising detecting CD25 in a sample obtained from a subject and selecting the subject for treatment with an anti-CD25-ADC if CD25 is expressed in cells in the sample.

3. The method of claim 1 or claim 2 wherein the expression of CD25 is determined to be overexpressed in the sample.

4. The method of any one of the preceding claims wherein 10% or more of the cells in the sample express CD25.

5. The method of any one of the preceding claims wherein the sample is a lymph node biopsy and 10% or more of the lymphocytes in the sample express CD25.

6. The method of any one of the preceding claims wherein at least 10% of the sample is determined to exhibit grade 1 staining for CD25.

7. The method of any one of claims 1 to 4 wherein the sample contains at least 500 pg/ml CD25.

8. The method of any one of the preceding claims wherein expression of CD25 in the cells in the sample is determined by immunohistochemistry.

9. The method of claim 8 wherein the sample is a formalin fixed paraffin embedded (FFPE) sample.

10. The method of any one of the preceding claims wherein the subject has, is suspected as having, is at risk of having, or has received a diagnosis of, cancer.

11. The method of claim 10 wherein the cancer is lymphoma.

12. The method of any one of the preceding claims wherein the sample is a sample of lymphoid tissue, tumor tissue, blood, plasma, serum or lymph.

13. The method of claim 5 wherein the lymphocytes are selected from one or more of activated T cells, activated B cells, thymocytes, myeloid precursors, oligodendrocytes, or tumor infiltrating lymphocytes.

14. A method comprising administering an anti-CD25-ADC to a subject who has been determined to be suitable for, or selected for, treatment using a method according to any one of the preceding claims.

15. A method comprising administering an anti-CD25-ADC to a subject, the subject having been determined to overexpress CD25.

16. An anti-CD25-ADC for use in a method of treatment in a subject selected for treatment using a method according to any one of claims 2 to 13.

17. An anti-CD25-ADC for use in a method of treatment in a subject, the subject having been determined to overexpress CD25.

18. Use of an anti-CD25-ADC in the manufacture of a medicament for the treatment of cancer in a subject selected for treatment using a method according to any one of claims 2 to 13.

19. Use of an anti-CD25-ADC in the manufacture of a medicament for the treatment of cancer in a subject, the subject having been determined to overexpress CD25

20. The method of any one of claims 1-15, or the anti-CD25-ADC for use according to claim 16 or claim 17, or the use according to claim 18 or claim 19, wherein the anti-CD25-ADC is ADCT-301.

21. Use of an anti-CD25 antibody for determining the suitability of a subject for treatment with an anti-CD25-ADC.

22. Use according to claim 21, wherein the anti-CD25 antibody is antibody 4C9.

23. A method comprising determining, in a sample obtained from a subject diagnosed with lymphoma, the level of expression of CD25 and, based on the level of CD25 determined, determining whether the patient is suitable for treatment with an anti-CD25-ADC and, if the patient is determined to be suitable for treatment with an anti-CD25-ADC, administering an anti-CD25-ADC to the patient.

24. A method comprising administering an anti-CD25-ADC to a subject determined to be suitable for such treatment using a method according to any one of claims 1 to 13, wherein the dosage of anti-CD25-ADC is selected based on the level of CD25 expressed in the sample.

25. A method comprising:
performing an immunohistochemical analysis of a sample from a subject, the sample having been fixed and incubated with an anti-CD25 antibody; and
determining the expression of CD25 in the sample; and
selecting a patient for treatment with anti-CD25-ADC based on the expression of CD25 in the sample.


SEQUENCE LISTING (8 sequences)

SEQ ID NO: 1 - protein, 127 aa, Homo sapiens
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLA

SEQ ID NO: 2 - protein, 119 aa, Homo sapiens
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFP

SEQ ID NO: 3 - protein, 5 aa, Homo sapiens
RYIIN

SEQ ID NO: 4 - protein, 17 aa, Homo sapiens
RIIPILGVENYAQKFQG

SEQ ID NO: 5 - protein, 6 aa, Homo sapiens
KDWFDY

SEQ ID NO: 6 - protein, 11 aa, Homo sapiens
RASQSVSSYLA

SEQ ID NO: 7 - protein, 7 aa, Homo sapiens
GASSRAT

SEQ ID NO: 8 - protein, 9 aa, Homo sapiens
QQYGSSPLT